(19) United States
(12) Patent Application Publication
(10) Pub. No.: US 2009/0018870 A1
(43) Pub. Date: Jan. 15, 2009

(54) Method for Preventing Chronic Disease

(76) Inventor: Joseph Thomas Weishaar, New York, NY (US)

Correspondence Address: JOSEPH THOMAS WEISHAAR, 501 W. 120TH ST., # 8-W, NEW YORK, NY, 10027, US

(21) Appl. No.: 12/281,259

(22) PCT Filed: Nov. 26, 2007
(86) PCT No.: PCT/US2007/085488

§ 371 Date: Aug. 29, 2008

Related U.S. Application Data
(60) Provisional application No. 60/872,271, filed on Dec. 1, 2006.
(60) Provisional application No. 60/917,760, filed on May 14, 2007.

(51) Int. Cl. G06Q 50/00; G06Q 30/00; G06Q 40/00; G01N 33/53

(52) U.S. Cl. 705/2; 705/14; 705/4; 436/129

(57) ABSTRACT

A new method for preventing chronic disease is described. The method comprises the act of using the result of any biometric test for an individual's levels of omega-6 or omega-3 fatty acids as an input in any computerized analysis that determines an economic outcome for the tested individual. This outcome will encourage improved health behavior, which will lead to lowered incidence of chronic disease, improved physical and mental health, improved employee performance, and lower health care costs.

### TECHNICAL FIELD

The field of this invention is methods for insurance, health benefits, or wellness programs.

### BACKGROUND ART

This invention is based upon the general method of using a test result in an analysis. The number of possible embodiments of this general method approaches infinity. The general method has been in use for tens of thousands of years. An example of a pre-historic embodiment of this method would be an early hominoid using the result of visual test of water clarity in an analysis to determine whether the water was drinkable. This example is a purely mental exercise. In other embodiments of this general method, however, the analysis is computerized, making the method more than a mental exercise and subject to patent if the specific method is useful, novel, and unobvious.

The present invention uses the result of a specific kind of biometric test, embodiments of which are known in the prior art, with a specific kind of computerized analysis, embodiments of which are known in the prior art. The usefulness of this invention is that it can be used to prevent chronic disease. The novelty of the invention is that no one had ever suggested in print or by patent the method of using a result from a biometric test of this specific kind as an input to a computerized analysis of this specific kind before this invention was reduced to practice by the present inventor by means of a provisional patent application on Dec. 1, 2006. Moreover, as this application will show, the invention isn't obvious. Prior art teaches against using the result of this specific kind of biometric test in this specific kind of analysis. Moreover, obtaining a biometric test result for each individual in a large group using today's commercially available testing technology would be considered cost prohibitive by anyone skilled in the field.

The specific kind of biometric test result used by the invention measures an individual's levels of omega-6 or omega-3 fatty acids.

The specific kind of analysis used by the invention is a computerized analysis that determines a direct economic outcome for the tested individual—for example, the cost, availability, or incentives of insurance, health benefits, or wellness programs.

Fatty acids: The secret of life is that fat and water don't mix. Fat or lipid molecules typically consist of a glycerol that has strings of carbon atoms attached to it. Imagine these strings of carbon atoms as strands of pearls. The strands connect at one end to the glycerol while the other ends float free.

Fat molecules can have one (monoglycerides), two (diglycerides), or three (triglycerides) of these strands of carbon atoms. When one of these strands of carbon atoms is disconnected from a glycerol it is called a fatty acid—creating a distinct difference between, for example, a “triglyceride” and a “fatty acid.” The end of the fatty acid strand that connects to the glycerol is called the carboxyl or delta end. The loose end of the strand is called the omega end.

Living organisms have enzymes that can convert starches into fatty acids, which are used to store energy for future use. In animals, fatty acids destined to be used for energy storage are connected to a glycerol in the form of a triglyceride. These triglycerides are stored in adipose cells, which are specialized fat-storage cells. Blood levels of triglycerides, but not fatty acids, are measured as part of standard cholesterol screening tests.

Organisms also use fatty acids to form membranes. One or two fatty acid strands, phosphorus, and other atoms connect to form a phospholipid. The phospholipid is water attracting near the phosphorus atom, while the fatty acid strands are water repelling. Consequently, these fats naturally organize as membranes. The waterrepelling fatty acids stick together on the inside of the membrane and the glycerols form the membrane's inner and outer faces. These lipid-based membranes provide the compartments that allow organic chemical reactions to proceed in isolation from each other. They form the skin of both cells and of the components within cells. Without fats there would be no membranes. Without membranes there would be no life.

Chemically, carbon atoms have four bonding points with adjacent atoms. Carbon atoms in the middle of a fatty acid strand connect to the prior and the subsequent carbon atoms and to two hydrogen atoms. The carbon atom on the end of the strand that's connected to the glycerol, the delta end, bonds to two oxygen atoms—one with a double bond, one with a single bond—and the second carbon atom in the strand. The carbon atom on the loose end of the strand, the omega end, connects to the prior carbon atom and three hydrogen atoms.

Carbon atoms can also connect to each other with double bonds. When this happens in a fatty acid strand, the two carbon atoms participating in the double bond each lose a hydrogen atom. A double carbon bond also forces all the participating atoms—the two double-bonded carbons, each connected by single bonds to a hydrogen atom and the strand's prior or subsequent carbon atom—to lie in a plane. If the two remaining hydrogen atoms are on the same side of the strand, this puts a bend or kink in the strand. This is called a cis configuration. If the hydrogen atoms are on opposite sides of the strand, on the other hand, the double bond strengthens the straightness of the fatty acid strand. This is called a trans configuration. Fatty acids in this configuration are called trans fats.

The enzymes that create double bonds are called desaturase. The mammalian genome codes only for desaturase that creates double bonds in the cis configuration, although desaturase that creates trans configurations occurs in other organisms. These natural trans fats appear in small quantities in human food supplies. Likewise, mammalian desaturase always creates double bonds that are spaced with a single carbon atom between the carbon atoms that are participating in the double bonds. This is called methyl interruption. Other organisms can create double bonds that are adjacent to each other, which are called conjugated. Conjugated fatty acids also appear in small amounts in the human food supply.

When fatty acid strands have no cis double-bond kinks, they tend to stick together. The longer the fatty acid strands, the stickier they are. Thus fats that contain pre-dominantly long strands are more solid and have a higher melting point. When some of the fatty acids have one or more cis double-bond kinks, on the other hand, the stickiness is disrupted. Cis double bonds lower a fat's melting point, making it more liquid. In addition, the cis double-bonds make a fat more chemically reactive. The more cis double bonds a fatty acid has, the more chemically reactive it becomes. Light, heat, and oxygen make fats rancid by creating reactions that attack the double bonds.

A fatty acid with no double bonds is called a saturated fat. It's saturated with the maximum number of hydrogen atoms. Saturated fats are solid at room temperature. A fatty acid with one double bond is called monounsaturated. Monounsaturated fat is liquid but cloudy in a refrigerator. A fatty acid with more than one double bond is called polyunsaturated. Polyunsaturated fat is liquid in a refrigerator.

An industrial process called hydrogenation can be used to convert the cis configurations in unsaturated fats into trans configurations. Removing the kinks makes the fat more solid and more stable. Fully hydrogenated fats have no double bonds left and thus are just saturated fats. In partially hydrogenated fats, however, most of the cis double bonds have been converted to trans double bonds. These industrially-created trans fats are unsaturated, yet with higher melting points and more chemical stability than natural unsaturated fats. They were introduced in the early twentieth century, but have recently been recognized as hazardous to human health and are being slowly removed from food supplies.

Fatty acids are characterized by the number of carbon atoms in the strand, the number of double bonds, the position of the double bonds, and the configuration of the double bonds. The fatty acids of interest to the human diet and human biochemistry all have an even number of carbon atoms. They range from four to 24 carbon atoms in length.

The fats that vertebrates, including humans, synthesize from starch have 16 carbon atoms. An enzyme called elongase can add pairs of carbon atoms to these fatty acids to create longer strands. Fatty acids can also be shortened by two carbon atoms in a process called peroxisome oxidation and they can be completely consumed by mitochondria to create energy.

Mammals, including humans, have desaturase enzymes that can place cis double bonds five, six, or nine carbon atoms from the delta end of a fatty acid strand. When delta-9 desaturase acts on a 16-carbon saturated fatty acid, the resulting fatty acid has one cis double bond that's nine carbon atoms from the delta end of the strand and seven carbon atoms from the omega end. This is called an omega-7, or n-7, fatty acid. The same desaturase acting on an 18-carbon fatty acid will create an omega-9, or n-9, fatty acid. The mammalian genome doesn't code for a desaturase that will create fatty acids with an omega number lower than seven.

Nonetheless, mammalian biochemistry, like the biochemistry of all other organisms, is dependent on fatty acids with a final double bond six carbon atoms or three carbon atoms away from the omega end of the strand. These are known as omega-6, or n-6, and omega-3, or n-3, fatty acids. Vertebrates, including humans, obtain these fatty acids by eating plants or an animal that has eaten plants. Because vertebrates must get these fatty acids through the diet, they are called “essential” fatty acids.

A simplified nomenclature for fatty acids gives the number of carbon atoms, the number of double bonds, and the position of the final double bond. For example, 18:3 n-6 describes the 18-carbon, three-double-bond, omega-6 fatty acid that's also known as gamma-linolenic acid or GLA. Unless otherwise specified, this nomenclature assumes the double bonds are cis rather than trans. It also assumes the double bonds are methyl interrupted rather than either conjugated or separated by more than one carbon.

The most common omega-6 and omega-3 fatty acids in plants are 18:2 n-6, linoleic acid (LA), and 18:3 n-3, alpha-linolenic-acid (ALA). Mammalian desaturase and elongase enzymes act sequentially to convert LA and ALA into a large variety of omega-6 and omega-3 fatty acids. These have from 18 to 24 carbons and from two to six double bonds. The same enzymes work with both omega-6 and omega-3 fatty acids, which means that if one type of fatty acid is present in overabundance, it will block the other from access to the enzymes and create a deficiency of longer chain fatty acids of the blocked type.

The most notable among these long chain fatty acids are the 20-carbon 20:3 n-6, dihomo-gamma-linolenic acid (DGLA), 20:4 n-6, arachidonic acid (AA), and 20:5 n-3, eicosapentaenoic acid (EPA), which are required to create intercellular hormones called eicosanoids, and the 22-carbon, six-double bond 22:6 n-3, docosahexaenoic acid (DHA), which appears in large quantities in brain, nerve, and eye tissues.

Today's commercially available biometric tests for determining an individual's levels of fatty acids are time- and material-intensive and costly on a per-sample basis. Lipids are extracted from bodily substances using specific kinds of solvents. The separated lipids are then analyzed using one or a combination of adsorption chromatography, thin-layer choromotography, gas-liquid chromatography, high-performance liquid chromatagraphy, or mass spectrometry.

Disease prevention initiatives are classified as primary, secondary, and tertiary. Because the present invention applies to entire populations rather than only those at risk for (secondary prevention) or having (tertiary prevention) a chronic disease, it is a primary prevention measure. However, art in this area teaches that a primary prevention measure must be inexpensive. Because the currently commercialized fatty acid testing methodologies are five to ten times more expensive than the biometric tests used in today's standard primary prevention initiatives, the art teaches away from using their results in a primary prevention initiative to prevent chronic disease (see: Biometric screening in health promotion: Is it really as important as we think?, by Chapman, L S, in the American Journal of Health Promotion Vol 7, No 2, pages 1-9, May-June 2003).

Economic outcome analysis: There are a large number of methods in the prior art for conducting economic outcome analysis. For example, the price and availability of insurance is currently determined primarily by using an actuarial analysis based on factors such as age, sex, smoking status, and preexisting disease conditions. Actuaries use these factors to make a mathematical projection of future health care costs or life expectancy for an individual or group of individuals seeking insurance. These actuarial projections are then used to determine the direct economic outcomes of whether insurance will be offered to a specific individual and at what cost.

In addition, non-actuarial analytic processes are used with health benefit and wellness programs to determine economic outcomes, such as incentives for individuals who modify their health behavior and disincentives for those who don't modify their health behavior.

U.S. Pat. No. 4,975,840—Method and apparatus for evaluating a potentially insurable risk—describes a method for evaluating the insurability of a risk. However, the patent does not mention or make a claim related to fatty acids.

U.S. Pat. No. 7,024,369—Balancing the comprehensive health of a user—describes an invention that prioritizes possible health behaviors based on an individual's current health profile, which includes multiple monitored physical parameters and multiple monitored environmental parameters. The invention aids the user in selecting from among possible health behaviors and in some embodiments provides incentives to users who adopt suggested behaviors. However, the patent does not mention or make a claim related to fatty acids.

US Patent Application 20020055857—Method of assisting individuals in lifestyle control programs conducive to good health—describes a system that includes a database of lifestyle data with correlations to good health and a personal logging device that collects the corresponding lifestyle data of an individual. The system produces correlations of the individual's lifestyle data with good health and provides the individual with health-promoting information based on the correlations. However, the application does not provide an economic outcome or mention fatty acids.

US Patent Application 20020103680—Systems, methods and computer program products for managing employee benefits—describes an invention for managing the provision and administration of employee benefits. The invention can analyze employee benefits based on detailed claim records, determine the level of discounts or the effective rate of discounts provided by various health care networks, generate health care and benefits usage pattern information for employees and their family members, perform disruption analysis to employees and their families caused by switching health care networks, forecast the effects of changes to group health insurance benefits plans, and compare actual usage of health care services by employees and their families against actuarial normative information to determine usage patterns. However, the application does not mention or make a claim related to fatty acids.

US Patent Application 20030065561—Providing consumers with incentives for healthy eating habits—describes a method, system, and program for providing consumers healthy habits incentives. The system determines health factors for various kinds of food that are available. An electronic incentive is selected for the individual according to the health factor, such that the individual is encouraged to consume a healthy selection of consumable items. An electronic incentive may be further specified according to the personal health history, exercise history, and consumption history of an individual. However, the application does not mention or make a claim related to fatty acids.

US Patent Application 20030135391—Method and system for analyzing health information—describes a method and system configured to establish a repository of health related information. The health related information may be used to manage a health care program. The method includes the steps of establishing a population and establishing the repository of health related information, to include a health characteristic, a family health characteristic, and a lifestyle characteristic associated with members of the population. However, the application does not mention or make a claim related to fatty acids.

US Patent Application 20040225200—System and method of analyzing the health of a population—describes a system that includes the steps of establishing health-related characteristics associated with a population, establishing a relationship between the health-related characteristics and specific health conditions, and analyzing the population in response to the relationship. The analysis may include establishing a characteristic associated with a health condition, applying an intervention in response to the characteristic, and monitoring the success of the intervention. However, the application does not mention or make a claim related to fatty acids.

US Patent Application 20040267565—Interactive system for tracking and improving health and well-being of users by targeted coaching—describes a method for expert health coaching of individuals to improve their health, fitness, and wellness. Using the Internet, online prompts are presented to the user to complete a daily questionnaire on a variety of health behaviors, including exercise, nutrition, sleep habits, and stress management. The data from the daily questionnaire are captured in a database and a daily score is calculated based on the input and weighted criteria. A coach is assigned to each individual and the personally assigned coach tracks the assigned individuals' progress daily and provides positive reinforcement. However, the application does not mention or make a claim related to fatty acids.

US Patent Applications 20050038679 and 20050010439—Method of promoting employee wellness and health insurance strategy for same—describes supplemental health insurance with benefits that are conditional on the insured voluntarily participating in a wellness program. The voluntary wellness program can include aspects such as screening tests, wellness education, disease inoculation, and injury prevention. The invention provides an incentive for employees to make healthier lifestyle choices. However, the application does not mention or make a claim related to fatty acids.

US Patent Application 20050102172—System and method for evaluating insurance member activity and pricing insurance products—describes systems and methods for evaluating and establishing pricing of insurance products based on insured member compliance to health-promoting measures. A member's participation in health-promoting measures is monitored and used as a basis for establishing incentives or reductions in insurance premiums. However, the application does not mention or make a claim related to fatty acids.

US Patent Application 20060064331—Insurance premium refund incentive program—describes a health insurance premium refund incentive program for consumers. Under the program, a portion of the premium can be refunded to consumers whose health behavior tends to result in reduced claims. However, the application does not mention or make a claim related to fatty acids.

US Patent Application 20060129436—Method of reducing employer health related costs while promoting employee wellness and health benefit plan strategy for same describes a voluntary employee wellness program wherein employees are rewarded for improving their health status in certain wellness categories. Wellness categories mentioned in the application include a tobacco-free category, a normal blood pressure category, a regular exercise category, a non-overweight category, a healthy cholesterol level category, a healthy blood glucose level category, and a category for participation in a health risk assessment. However, the application does not mention or make a claim related to fatty acids.

U.S. Pat. No. 6,059,724—System for predicting future health—describes a computer-based system for predicting the future health of an individual. It obtains data over time for a large number of biomarkers from a large human test population, statistically selects predictive biomarkers, and determines and assesses appropriate statistical functions based upon the selected biomarkers. A table in this patent lists a very large number of potential biomarkers, including several fatty acids that are used by the present invention. However, this patent covers a system for discovering what biomarkers might be relevant to health. It does not pretend to already know what it might discover. Moreover, it makes no assertions related to economic outcomes or incentives for tested individuals.

US Patent Application 20050177397—Network and methods for integrating individualized clinical test results and nutritional treatment—describes a system in which a variety of biomarkers, including the results of tests for fatty acids like those required by the present invention, are used to provide individualized nutritional diagnosis and treatment to consumers. However, the application makes no assertions related to economic outcomes or incentives for tested individuals.

US Patent Application 20070118398—System and method for estimating life expectancy and providing customized advice for improving life expectancy—describes a system that estimates a person's life expectancy and provides customized advice for improving it. The food survey specified by this system could be considered a test for fatty acid levels, but it's not the type of biometric test required by the present invention. Likewise, the health outcome of an increase in life-expectancy could be considered an incentive, but it's not the type of economic outcome required by the present invention.

US Patent Applications 20050228692 and 20050234742, as well as International Patent Application PCT/US2005/010489—Incentive based health care insurance program—describes a system of offering incentives to a participant of a health care program to complete at least one of a health risk assessment questionnaire, a biometric measurement analysis, or a biosample for biomedical analysis. The measure is used to determine a Health Score that describes the presence or risk of disease. The system optionally provides incentives to the participants to improve their Health Score.

Paragraph 46 of the detailed description of this application says, “Incentives can also be offered to a participant to implement a dietary change, which includes, for example . . . omega 3 fatty-acid supplementation . . . and limiting intake of trans fatty acids.” Making such a dietary change could, over time, improve the results of the tests that are used to implement the present invention. However, the application doesn't suggest testing levels of fatty acids in either the specification or the claims. Claims 23 and 24 specify a large number of “biomedical parameters”, including triglycerides, however, the list doesn't include a any type of fatty acid. Triglyceride levels are a component of standard cholesterol screening tests and provide no information about levels of specific fatty acids.

Hundreds of other patents or patent applications mention omega-3 fatty acids or particular omega-3 fatty acids. However, most of these are in the field of public health and none disclose the present invention. The inventions describe specific nutritional supplements or other compositions or compounds, therapeutic methods for preventing or treating particular diseases, or components of biochemical reactions. The present invention is in a different field, relates to economic outcomes for individuals, and is a method rather than a material substance.

Prior art in medical literature: Before the present invention could be obvious to someone with ordinary skill in the field of methods for insurance, health benefits, or wellness programs, the usefulness of testing an individual's levels of specific fatty acids must first be obvious to the medical profession. Medical advances flow from medical researchers to medical associations and government agencies, then on to individual doctors and providers of insurance, health benefits, or wellness programs.

Using fatty acid levels as a biomarker relevant to health has not been accepted as a viable practice by any medical association or government agency. Those skilled in the art of providing insurance, health benefits, or wellness programs do not consider such testing viable for two reasons: limited scientific evidence of its usefulness and the high cost of such testing.

As of Dec. 1, 2006—the date the present invention was reduced to practice by means of a provisional patent application—no medical association or government agency recommended testing an individual's levels of specific fatty acids. Health insurance companies reconfirmed their lack of interest in fatty acid levels by refusing to reimburse for this type of testing.

US Department of Health and Human Services: As of the date of the provisional patent application on which the application is based, the Agency for Healthcare Research and Quality of the US Department of Health and Human Services had released a series of reports on the effects of omega-3 fatty acids on a range of diseases.

The most recent report on asthma concludes, “Aside from an acceptable safety profile, it is impossible to definitively conclude anything with respect to the value of using omega-3 fatty acid supplementation in asthma for adults or children, either in or beyond North America. The lack of sufficiently consistent evidence—as well as a paucity of evidence from well-designed, well-conducted, and adequately powered studies—suggests that no definitive conclusion can yet be drawn regarding the efficacy of omega-3 fatty acid supplementation as an asthma treatment. Future research investigating North American samples is likely to be needed to establish or refute the value of omega-3 fatty acids in the treatment or prevention of asthma in North American adults and children.”

The most recent report on cardiovascular disease [CVD] concludes, “Overall, consumption of omega-3 fatty acids, whether from fish or from supplements, reduces all-cause mortality and improves various CVD outcomes. The evidence for benefit from ALA supplements is sparse and inconclusive. The adverse events due to consumption of fish oil or ALA supplements, most commonly gastrointestinal symptoms, appear to be minor. It may not be appropriate to apply the results of the non-U.S. population studies to the U.S. population. Future research needs to address optimal quantity and type of omega-3 fatty acid, ratio to omega-6 fatty acids, the effect of different fish and types of preparation, and the needs of different subpopulations.”

The most recent report on arrhythmogenic mechanisms concludes, “ . . . Fish oil supplementation (EPA and/or DHA) might have anti-arrhythmic effects when compared with omega-6, monounsaturated, or saturated fatty-acids in pre-fed fish oil in studies of various animal species. Fish oil supplements in rats showed significant protective effects for ischemia- and reperfusion-induced arrhythmias by reducing the incidence of ventricular tachycardia and fibrillation but no beneficial effects for ALA supplementation were found. The arrhythmic effects for infused omega-3 fatty-acid treatments are still unknown . . . . ”

The most recent report on type II diabetes, rheumatoid arthritis, and other diseases concludes, “The evidence for effects of omega-3 fatty acids on outcomes in the conditions assessed varies greatly. Omega-3 fatty acids appear to reduce serum triglycerides among type II diabetics, but have no effect on total cholesterol, HDL cholesterol, and LDL cholesterol. There appears to be no effect on most clinical outcomes in rheumatoid arthritis, although tender joint count may be reduced. There are insufficient data to draw conclusions about IBD [irritable bowel disease], renal disease, SLE [systemic lupus erythematosis], bone density or fractures, requirement for anti-inflammatory or immunosuppressive drugs, or insulin resistance among type II diabetics.”

The most recent report on cancer concludes, “In a large body of literature spanning many countries and cohorts with different demographic characteristics, the evidence does not suggest a significant association between omega-3 fatty acids and cancer incidence. In a small body of literature, there is no significant association between omega-3 fatty acids and clinical outcomes after tumor surgery. Although the combination of omega-3 fatty acids, arginine, and RNA are associated with a reduced risk of postoperative complications and reduced length of hospital stay, it is not possible to ascertain whether these effects are due to omega-3 fatty acids, arginine, RNA, or a combination of these. A large body of literature (of varying quality) suggests that omega-3 dietary enrichment may help inhibit or prevent tumor growth in some animal models; the quality of the review articles is not, however, sufficient to permit drawing strong conclusions.”

The most recent report on cognitive functions concludes, “The quantity and strength of evidence for effects of omega-3 fatty acids on the neurological conditions assessed vary greatly. The small number of studies that met the inclusion criteria suggest that a great deal of epidemiological and clinical research remains to be done before any strong conclusions can be drawn or policy recommendations can be made regarding the neurological health effects of omega-3 fatty acids.”

The most recent report on eye health concludes, “Based on the studies identified by this review, clinical research has only scratched the surface with respect to understanding the possible utility of the intake of omega-3 fatty acids as a primary or secondary prevention in eye health . . . . There is little understanding of the exact sources, types, and doses of omega-3 fatty acids, or even the possible duration of their use, which might usefully serve as definitions of a prevention-centered ‘intervention’ for any of the eye diseases/visual impairments examined in our review . . . . More research is needed with respect to the effects of omega-3 fatty acids on eye health. Also, determining the possible benefits of the intake of omega-3 fatty acids in eye health might profit from considering the impact of the concurrent intake of omega-6 fatty acids and, by definition, the omega-6/omega-3 fatty acid intake ratio.”

The most recent report on mental health concludes, “Only with respect to the supplemental treatment of schizophrenia is the evidence even somewhat suggestive of omega-3 fatty acids' potential as short-term intervention . . . . Nothing can yet be concluded concerning the clinical utility of omega-3 fatty acids as supplemental treatment for any other psychiatric disorder or condition, or as a primary treatment for all psychiatric disorders or conditions, examined in our review. Primary treatment studies were rare. Because of limited study designs, little is known about the relationship between PUFA [polyunsaturated fatty acid)] biomarker profiles and the onset of any psychiatric disorder or condition. Studies examining the possible association between the intake of omega-3 fatty acids, or the PUFA content of biomarkers, and the continuation or recurrence of psychiatric disorders or conditions were virtually nonexistent. Future research investigating North Americans populations or those exhibiting a high omega-6/omega-3 fatty acid intake ratio similar to what has been observed in the diet of North Americans will likely be needed. It may behoove researchers to investigate the possible therapeutic or preventive value of the dietary omega-6/omega-3 fatty acid intake ratio.”

The most recent report on child and maternal health concludes, “ . . . Results concerning the impact of the intake of omega-3 fatty acids on the development of infants are primarily, although not uniformly, inconclusive. The inconsistencies in study results may be attributable to numerous factors. Reliably ascribing definite child outcome-related benefits, or the absence thereof, to specific omega-3 fatty acids is difficult. Biomarker data failed to clarify patterns in child outcome data. Future research should likely consider investigating the impact of specific omega-6/omega-3 fatty acid intake ratios . . . . ”

The most recent report on organ transplantation concludes, “ . . . Inconsistent benefits on renal function across studies may suggest a potential benefit in a subset of patients, the characteristics of whom remain unclear. Whether administration of omega-3 fatty acids prior to transplantation would improve its benefits is unclear. Long-term studies are needed to determine whether benefits on renal function translate into improved renal outcomes. Similarly, long-term follow-up in recipients of heart transplants will be required to determine whether fish oil supplementation reduces the risk of post-transplant atherosclerosis. Because of the scarcity of data, the effects of ALA supplementation in the transplant setting cannot be determined . . . . ”

US Food and Drug Administration: Since Sep. 8, 2004, the US Food and Drug Administration (FDA) has allowed a qualified health claim for reduced risk of coronary heart disease on conventional foods and dietary supplements that contain the omega-3 fatty acids EPA or DHA. The FDA does not suggest testing the levels of fatty acids in individuals.

Medical Associations In February 2006, the American Academy of Pediatrics issued an “Endorsed Policy Statement” on dietary recommendations for children and adolescents. The statement recommends an increased emphasis “on ensuring adequate intakes of omega-3 fatty acids,” but doesn't suggest testing the levels of fatty acids in individuals.

On Jun. 19, 2006, the American Heart Association (AHA) published an “AHA Scientific Statement” that recommends eating oily fish twice a week because “the consumption of 2 servings (8 ounces) per week of fish high in EPA and DHA is associated with a reduced risk of both sudden death and death from coronary artery disease in adults.” The AHA also recommends omega-3 dietary supplements for patients who have elevated triglycerides. However, the association doesn't suggest testing the levels of fatty acids in individuals.

On Dec. 11, 2006, the American Geriatrics Society released a tip sheet that “explains what older adults need to do to keep their cognitive edge. Among other things, older people should . . . eat a diet that's low in saturated fat and includes plenty of fruits and vegetables and two servings of fish rich in omega-3 fatty acids weekly.” The society doesn't suggest testing the levels of fatty acids in individuals.

On Apr. 1, 2007, the American Academy of Family Physicians released guidelines for the prevention of cardiovascular disease in women, noting that, “in conjunction with diet, omega-3 fatty acids in capsule form may be considered for women with coronary heart disease.” The academy doesn't suggest testing the levels of fatty acids in individuals.

Economic analysis in medical research: Studies have been conducted to estimate the cost-effectiveness of treating patients with omega-3 supplements for heart disease. For example, see Cost-Effectiveness Analysis of n-3 Polyunsaturated Fatty Acids (PUFA) after Myocardial Infarction, by Maria Grazia Franzosi et al in Pharmacoeconomics 2001; 19 (4): 411-420. These analyses, however, don't determine an economic outcome for the tested individual, but an economic outcome for a third-party payer based on the treatment group the individual is in.

Other prior art: While a person with ordinary skill in the art of medicine currently sees no utility in testing individuals for specific levels of fatty acids, research in biochemistry, molecular biology, and a few medical settings has shown so much potential for optimizing an individual's levels of fatty acids that a few individuals have begun to recommend such testing to improve health outcomes. Prominent among these people is Dr. Barry Sears, who developed the Zone Diet.

In his book, The Anti-Inflammation Zone, published on Dec. 13, 2005, Sears explains why a proper dietary balance of omega-3 and omega-6 fatty acids is important. Sears recommends that individuals use tests of their personal fatty acid levels to determine if their dietary intake of these fatty acids is appropriate.

This book was published less than a year before the date of the provisional patent application associated with the present invention, which disqualifies it as prior art. In a chapter of this book, called “Avoiding the Coming Collapse of the Health Care System” (pages 307-315), Sears suggests paying physicians based on the results of the fatty acid tests of their patients and recommends that corporate wellness programs use these tests to determine their effectiveness. Neither of these suggestions disclose the present invention, which uses these tests to determine an economic outcome for the individual being tested, not an economic outcome for the individual's physician or for the organizers of the individual's wellness program.

The prior art does not anticipate the present invention. A professional of ordinary skill in the field of methods for insurance, health benefits, or wellness programs might find the present invention to be “unlikely,” “too expensive,” or “premature,” but certainly not “obvious.”

### DISCLOSURE OF INVENTION

The invention is a method that improves current methods for determining direct economic outcomes for individuals in programs such as insurance, health benefits, or wellness programs. The method consists of using the result of one or more biometric tests of an individual's levels of omega-6 or omega-3 fatty acids as an input to a computerized analysis that determines a direct economic outcome for that individual.

Although the present invention relies upon the result of a biometric test for levels of omega-6 or omega-3 fatty acids, the invention is agnostic to the methodology of this test. The methodology could include analysis of any bodily substance, excretion, or exhalation using, for example, adsorption, thin-layer, gas-liquid, or high-performance liquid chromatography; mass spectrometry; magnetic resonance imaging; broad spectrum absorbance and/or fluorescence measurements—e.g. U.S. Pat. No. 6,737,275; or any other direct or indirect biometric test reporting omega-6 or omega-3 fatty acid levels or the equivalent by another name. What the present invention requires is only the result of the test. The process used to arrive at this result is a series of steps that occur before the present invention is employed.

The result provided by the biometric testing methodology might be a result for an individual or, using sampling techniques, a result for a group that includes the individual. Moreover, the result could report actual or estimated total omega-6 or total omega-3 levels, levels of any or several of the specific omega-6 or omega-3 fatty acids, ratios of total or specific omega-6 and omega-3 fatty acids, or comparisons of total or specific omega-6 or omega-3 fatty acids to levels of other fatty acids.

The present invention is a method employed only with a specific kind of computerized analysis. This is an analysis that determines a direct economic outcome for the individual whose test result is used in the analysis. The invention is agnostic to the methodology used by the analysis. This methodology will be a series of steps, some of which may occur before the invention is employed and some that will occur after. The outcome of the steps that occur after the invention is employed will be influenced by the invention and will determine a direct economic outcome for the individual whose test result is used in the analysis.

Because the invention methodologically combines the result of any type of biometric fatty acid test with any type of analysis that determines a direct economic outcome for the tested individual, numerous embodiments of the invention are possible and would be obvious to anyone skilled in the art once the invention and its impact had been disclosed to that skilled individual. In general, these obvious embodiments would be customized for specific contexts by those skilled in the art rather than provided in a single embodiment intended for all contexts.

The best embodiment of the invention is the single act of including the test result as an input to an appropriate analysis.

### BRIEF DESCRIPTION OF DRAWINGS

Not Applicable

### MODES FOR CARRYING OUT THE INVENTION

In some embodiments the invention is the combination of the act of obtaining the test result and the act of including the test result as an input to an appropriate analysis.

In some embodiments the test result is used in an actuarial analysis.

In some embodiments the test result is used in an analytical process, a predictive model, or a statistical or other computerized analysis.

In some embodiments the test result reports levels of specific omega-6 fatty acids.

In some embodiments the test result reports levels of specific omega-3 fatty acids.

In some embodiments the test result reports the ratio of omega-6 to omega-3 fatty acids.

In some embodiments the test result reports the levels of omega-6 or omega-3 fatty acids using calculations based on the number of carbon atoms and the number of double bonds in the examined fatty acids.

In some embodiments the test result reports the mean or modal fatty acid levels in a group of individuals that includes the individual receiving the economic outcome, in others it reports the fatty acid levels of that specific individual.

In some embodiments the economic outcome of the analysis applies to the tested individual.

In some embodiments the economic outcome of the analysis applies to an individual, group, or organization that has taken on any level of responsibility for the health of the individual.

In some embodiments the economic outcome is the price or availability of health insurance.

In some embodiments the economic outcome is the price or availability of life insurance.

In some embodiments the economic outcome is the price or availability of disability insurance.

In some embodiments the economic outcome is the price or availability of long-term care insurance.

In some embodiments the economic outcome is the price or availability of health benefits.

In some embodiments the economic outcome is a discount, a higher or lower price, or a fee.

In some embodiments the economic outcome is an incentive or disincentive.

In some embodiments the economic outcome is the price or availability of past or future fatty acid tests.

In some embodiments the economic outcome is the price or availability of dietary fatty acid supplements.

In some embodiments the economic outcome is employment.

In some embodiments the invention is used to improve a method for evaluating the insurability of a risk, such as the system described in U.S. Pat. No. 4,975,840, cited above.

In some embodiments the invention is used to improve a method for calculating participant incentives, such as the system described in U.S. Pat. No. 7,024,369, cited above.

In some embodiments the invention is used to improve a method of providing consumers with incentives for healthy eating habits, such as the system described in US Patent Application 20030065561, cited above.

In some embodiments the invention is used improve a method of calculating incentives in a system for analyzing health information, such as the system described in US Patent Application 20030135391, cited above.

In some embodiments the invention is used to improve a system's method of promoting employee wellness and health insurance strategy, such as the system described in US Patent Applications 20050038679 and 20050010439, cited above.

In some embodiments the invention is used to improve a system's method of evaluating insurance member activity and pricing insurance products, such the system described in US Patent Application 20050102172, cited above.

In some embodiments the invention is used to improve the method of determining incentives in an insurance premium refund incentive program, such the system described in US Patent Application 20060064331, cited above.

In some embodiments the invention is used to improve a system's method of reducing employer health related costs while promoting employee wellness, such as the system described in US Patent Application 20060129436, cited above.

In some embodiments the invention is used to improve a system for estimating life expectancy and providing customized advice for improving life expectancy, such as the system described in US Patent Application 20070118398, cited above.

In some embodiments the invention is used to improve an incentive-based health care insurance program, such as the program described in US Patent Applications 20050228692 and 20050234742 and International Patent Application PCT/US2005/010489, cited above.

### ADVANTAGEOUS EFFECTS

There now exists evolutionary, epidemiological, and biochemical evidence showing that a deficiency of omega-3 fatty acids or an imbalance in the omega-6/omega-3 ratio is associated with all-cause mortality in both men and women; with many chronic diseases, including heart disease, cancer, diabetes, stroke, and depression; and with death by homicide and suicide.

From a biochemical perspective, we know that a large number of cell functions including membrane rigidity, enzyme and genome signaling, and creation of eicosanoids—are controlled by the relative concentrations of omega-6 and omega-3 fatty acids in cells. Those concentrations, in turn, are controlled by diet. During human evolution, diets are believed to have had a much lower ratio of omega-6 to omega-3 fatty acids than modern diets. Consequently, the normal ratio found in today's populations is not optimal. This imbalance can be corrected by limiting intake of omega-6 fats and supplementing the diet with omega-3 fats.

Correcting this imbalance will have a significant positive impact on human health. An epidemiological study (Healthy intakes of n-3 and n-6 fatty acids: estimations considering worldwide diversity, by Hibbeln J R, Nieminen L R G, Blasbalg T L, et al. in American Journal of Clinical Nutrition 83 (6): pages 1483S-1493S Suppl. S June 2006) comparing food consumption and death rates in different countries, associated omega-3 deficiencies with more than 20% of deaths for all-cause mortality in males, 30% for all-cause mortality in females, 40% for coronary heart disease, 30% for strokes, 25% for homicide, and 90% for major depression. Providing economic outcomes to individuals who adjust their diets to correct this imbalance could have immense positive implications for public health, for employee performance, and for the ever-rising cost of health care.

By providing a method to prevent chronic disease, the present invention can allow organizations to improve their operations by identifying and rewarding individuals who have a probability of ongoing good health and associated low health care costs. Use of the invention will encourage individuals to make lifestyle changes that will improve their personal test results. Improved test results will lead to better physical and mental health and lower health care costs. Thus the present invention has the potential to prevent chronic disease while both improving the performance and lowering the health care cost of the employees of any organization that incorporates the invention into its health insurance, health benefit, or wellness programs.

### GLOSSARY

In general, words or terms used herein mean what an American English speaker with ordinary skill in the art of science journalism would take them to mean. The word “or” in this document is always meant in its inclusive sense. For example, “a or b” should be interpreted as “any result selected from the group consisting of ‘a’, ‘b’, and ‘a and b’.” The following words have these meanings:

| | |
|---|---|
| Biometric test | any assay of levels of one or more materials |
| | of biological interest that involves the exam- |
| | ination of any bodily substance, excretion, or |
| | exhalation |
| Computerized analysis | any analysis that is completed with the aid of a |
| | man-made computing device |
| Direct economic outcome | a result of an analytic process that can impact |
| | material wealth, accomplishment, or enjoyment |
| | in a primary way, rather than in a secondary |
| | way that is a result of good health |
| Health benefit | a product or service related to paying for the |
| | maintenance or improvement of the physical or |
| | mental health of an individual or a group |
| Incentive | something intended to induce action by offering |
| | a positive or negative change in material |
| | wealth, accomplishment, or enjoyment to the |
| | incented individual. The word also encom- |
| | passes the concepts of discounts and dis- |
| | incentives |
| Insurance | any product, service, or government program |
| | that could be considered insurance or that is |
| | marketed as insurance |
| Wellness program | any system that aims to maintain or improve |
| | the health of participants, even if the program |
| | goes by another name, such as health program, |
| | health education program, health promotion |
| | program, disease prevention program, weight- |
| | loss program, nutritional program, or a trade- |
| | marked or common name |

1-20. (canceled)

21. A process for determining a direct economic outcome for an individual given a device comprising a computer with code operable to transform a biometric test result from the individual into the direct economic outcome for the individual; the process comprising:
(a) obtaining a biometric test result that measures a level of fatty acid within a sample of biological material from the individual, wherein the fatty acid is selected from a group consisting of omega-3 and omega-6;
(b) inputting the biometric test result from the individual into the device; and
(c) collecting the direct economic outcome for the individual from the device;
whereby the determined direct economic outcome collected from the device influences a health behavior of the individual, leading to improved physical and mental health and a lower rate of chronic disease.

22. The process of claim 21 further comprising the steps of:
(a) taking a sample of biological material from the individual; and,
(b) performing a biometric test on the sample to measure a level of fatty acid.

23. The process of claim 21 wherein the direct economic outcome is an incentive selected from a group consisting of a discount, a higher price, a lower price, and a fee.

24. The process of claim 21 wherein the direct economic outcome is selected from a group consisting of a cost for insurance and an availability of insurance.

25. The process of claim 24 wherein the insurance is health insurance.

26. The process of claim 24 wherein the insurance is life insurance.

27. The process of claim 24 wherein the insurance is disability insurance.

28. The process of claim 24 wherein the insurance is long-term care insurance.

29. The process of claim 21 wherein the direct economic outcome is selected from a group consisting of a cost for health benefits and an availability of health benefits.

30. The process of claim 21 wherein the direct economic outcome is selected from a group consisting of a wellness program incentive and a wellness program disincentive.

31. The process of claim 21 wherein the direct economic outcome is a decision on employment.

32. The process of claim 21 wherein the direct economic outcome is selected from a group consisting of a price for the fatty acid test, a price for a future fatty acid test, and an availability of a future fatty acid test.

33. The process of claim 21 wherein the direct economic outcome is selected from a group consisting of a price for a dietary fatty acid supplement and an availability of a dietary fatty acid supplement.

34. A process for determining a direct economic outcome for an individual given a device comprising a computer with code operable to transform a biometric test result from the individual into the direct economic outcome for the individual; the process comprising:
(a) obtaining a biometric test result that measures a level of fatty acid within a sample of biological material from the individual, wherein the fatty acid is selected from a group consisting of omega-3 and omega-6;
(b) a step for using the device to determine the direct economic outcome for the individual;
whereby the determined direct economic outcome influences a health behavior of the individual, leading to improved physical and mental health and a lower rate of chronic disease.
